SEQUENCE LISTING (7 sequences)

SEQ ID NO: 1 - DNA, 1248 bp, Homo Sapiens
catatgtccaacgaactgcaccaggttccatcgaactgtgactgtctaaatggaggaacatgtgtgtccaacaagtacttctccaacattcactggtgcaactgcccaaagaaattcggagggcagcactgtgaaatagataagtcaaaaacctgctatgaggggaatggtcacttttaccgaggaaaggccagcactgacaccatgggccggccctgcctgccctggaactctgccactgtccttcagcaaacgtaccatgcccacagatctgatgctcttcagctgggcctggggaaacataattactgcaggaacccagacaaccggaggcgaccctggtgctatgtgcaggtgggcctaaagctgcttgtccaagagtgcatggtgcatgactgcgcagatggaaaaaagccctcctctcctccagaagaattaaaatttcagtgtggccaaaagactctgaggccccgctttaagattattgggggagaattcaccaccatcgagaaccagccctggtttgcggccatctacaggaggcaccgggggggctctgtcacctacgtgtgtggaggcagcctcatcagcccttgctgggtgatcagcgccacacactgcttcattgattacccaaagaaggaggactacatcgtctacctgggtcgctcaaggcttaactccaacacgcaaggggagatgaagtttgaggtggaaaacctcatcctacacaaggactacagcgctgacacgcttgctcaccacaacgacattgccttgctgaagatccgttccaaggagggcaggtgtgcgcagccatcccggactatacagaccatctgcctgccctcgatgtataacgatccccagtttggcacaagctgtgagatcactggctttggaaaagagaattctaccgactatctctatccggagcagctgaaaatgactgttgtgaagctgatttcccaccgggagtgtcagcagccccactactacggctctgaagtcaccaccaaaatgctgtgtgctgctgacccacagtggaaaacagattcctgccagggagactcagggggacccctcgtctgttccctccaaggccgcatgactttgactggaattgtgagctggggccgtggatgtgccctgaaggacaagccaggcgtctacacgagagtctcacacttcttaccctggatccgcagtcacaccaaggaagagaatggcctggccctctaactcgag

SEQ ID NO: 2 - protein, 412 aa, Homo Sapiens
MSNELHQVPSNCDCLNGGTCVSNKYFSNIHWCNCPKKFGGQHCEIDKSKTCYEGNGHFYRGKASTDTMGRPCLPWNSATVLQQTYHAHRSDALQLGLGKHNYCRNPDNRRRPWCYVQVGLKLLVQECMVHDCADGKKPSSPPEELKFQCGQKTLRPRFKIIGGEFTTIENQPWFAAIYRRHRGGSVTYVCGGSLISPCWVISATHCFIDYPKKEDYIVYLGRSRLNSNTQGEMKFEVENLILHKDYSADTLAHHNDIALLKIRSKEGRCAQPSRTIQTICLPSMYNDPQFGTSCEITGFGKENSTDYLYPEQLKMTVVKLISHRECQQPHYYGSEVTTKMLCAADPQWKTDSCQGDSGGPLVCSLQGRMTLTGIVSWGRGCALKDKPGVYTRVSHFLPWIRSHTKEENGLAL

SEQ ID NO: 3 - DNA, 46 bp, Artificial Sequence
  Synthetic Construct
catatgtccaacgaactgcaccaggttccatcgaactgtgactgtc

SEQ ID NO: 4 - DNA, 30 bp, Artificial Sequence
  Synthetic Construct
ctcgagttagagggccaggccattctcttc

SEQ ID NO: 5 - DNA, 1239 bp, Artificial Sequence
  Synthetic Construct
atgagcaatgaactgcatcaggttccgagcaactgtgattgtctgaatggtggcacctgtgtgagcaacaaatacttcagcaacattcactggtgcaactgcccgaaaaaattcggtggccagcactgtgaaatcgataaaagcaaaacctgctatgaaggcaatggtcacttttaccgcggcaaagccagcaccgataccatgggccgtccgtgcctgccgtggaacagcgccaccgttctgcagcagacctaccatgcccaccgtagcgatgcgctgcagctgggcctgggtaaacataattactgccgcaacccggataaccgtcgtcgtccgtggtgctatgtgcaggtgggcctgaaaccgctggttcaggaatgcatggtgcatgattgcgcggatggtaaaaaaccgagcagcccgccggaagaactgaaattccagtgtggccagaaaaccctgcgtccgcgctttaaaattattggcggcgaattcaccaccatcgaaaaccagccgtggtttgcggccatctaccgtcgtcaccgtggtggcagcgttacctacgtgtgtggtggcagcctgatcagcccgtgctgggtgatcagcgccacccactgcttcattgattacccgaaaaaagaagattacatcgtttacctgggtcgcagccgtctgaacagcaacacccagggcgaaatgaaatttgaagtggaaaacctgatcctgcacaaagattacagcgcggataccctggcgcaccacaacgatattgccctgctgaaaatccgtagcaaagaaggccgttgtgcgcagccgagccgcaccatccagaccatctgcctgccgagcatgtataacgatccgcagtttggcaccagctgtgaaatcaccggctttggcaaagaaaatagcaccgattatctgtatccggaacagctgaaaatgaccgttgtgaaactgattagccaccgtgaatgtcagcagccgcactactacggcagcgaagtgaccaccaaaatgctgtgtgcggcggatccgcagtggaaaaccgatagctgccagggtgatagcggtggtccgctggtttgtagcctgcagtgccgcatgaccctgaccggtattgtgagctggggccgtggttgtgccctgaaagataaaccgggcgtttacacccgtgttagccacttcctgccgtggatccgcagccacaccaaagaagaaaatggcctggcactgtaa

SEQ ID NO: 6 - DNA, 33 bp, Artificial Sequence
  Synthetic Construct
atcactggctttggacacgagaattctaccgac

SEQ ID NO: 7 - DNA, 33 bp, Artificial Sequence
  Synthetic Construct
gtcggtagaattctcgtgtccaaagccagtgat

(19) United States
(12) Patent Application Publication
(10) Pub. No.: US 2007/0009506 A1
(43) Pub. Date: Jan. 11, 2007

(54) Methods for production of recombinant urokinase

(76) Inventor: Xinli Lin, Piedmont, CA (US)

Correspondence Address: MORRISON & FOERSTER LLP, 755 PAGE MILL RD, PALO ALTO, CA, 94304-1018, US

(21) Appl. No.: 11/444,594

(22) Filed: May 31, 2006

Related U.S. Application Data
(63) Continuation of application No. 10/825,911, filed on Apr. 16, 2004, now abandoned.
(60) Provisional application No. 60/463,632, filed on Apr. 16, 2003.
(60) Provisional application No. 60/498,134, filed on Aug. 26, 2003.

(30) Foreign Application Priority Data
Sep. 25, 2003 (CN) 03134847.5

(51) Int. Cl. A61K 38/48; C12N 9/72

(52) U.S. Cl. 424/94.64; 435/215

(57) ABSTRACT

Highly efficient methods of producing properly folded recombinant urokinase are provided. Denatured recombinant pro-urokinase is refolded by first solubilizing the protein with a chaotroph at high pH, followed by refolding in the presence of reduced concentrations of chaotroph while the pH is slowly reduced.

### CROSS-REFERENCES TO RELATED APPLICATIONS

This application is a continuation application of U.S. patent application Ser. No. 10/825,911, filed Apr. 16, 2004, which claims priority to U.S. provisional patent applications 60/463,632, filed Apr. 16, 2003, 60/498,134, filed Aug. 26, 2003, and Chinese patent application 03134847.5, filed Sep. 25, 2003, the disclosures of which are herein incorporated by reference in their entirety.

### STATEMENT REGARDING FEDERALLY SPONSORED RESEARCH OR DEVELOPMENT

This invention was made with U.S. Government support SBIR 1 GRANT 1 R43 HL075883-01. The U.S. Government may have certain rights in this invention.

### BACKGROUND OF THE INVENTION

Urokinase is a serine protease that cleaves plasminogen to produce active plasmin, thus playing a key role in fibrinolysis. Clinically, this activity has been exploited for the treatment of thrombosis, including thrombotic stroke, pulmonary embolus, deep vein thrombosis, and the like.

Urokinase is initially synthesized as a 411 amino acid precursor protein with 12 intrachain disulfide bonds. Pro-urokinase is activated to mature urokinase by a proteolytic event which clips the backbone after Lys₁₅₈, generating a disulfide-linked two-chain molecule. Urokinase produced for clinical applications is purified from collected urine, which raises safety and reproducibility concerns.

Recombinant forms of urokinase, including “low molecular weight urokinase” (which has the amino terminal 125 amino acids deleted) and other variants have been reported. See Orsini et al. (1991, Eur. J. Bioch. 195:691-97), Liu et al. (2002, Circ. Res. 90:757-63), Tang et al. (1997, Prot. Express. Purif. 11:279-83), Winkler et al. (1986, Biochem. 25:4041-45) and U.S. Pat. Nos. 5,188,829, 5,219,569, and 5,472,692.

Pro-urokinase, when expressed at high levels in bacteria, accumulates in the form of insoluble “inclusion bodies” or “refractile bodies.” The protein contained in these insoluble particles is misfolded, and must be ‘unfolded’ (denatured, including reduction of mis-paired disulfide bonds) before it can be folded into the correct conformation. A number of refolding protocols have been reported, including U.S. Pat. Nos. 4,511,503; 4,599,197; U.S. Patent Publication 2001/0044521 (U.S. Pat. No. 6,583,268); 2003/0199676; PCT WO 01/55174.

All patents, patent applications, and publications cited herein are hereby incorporated by reference in their entirety. It should be noted that reference to a publication in this Background section is not an admission that the publication constitutes prior art to the instant invention.

### BRIEF SUMMARY OF THE INVENTION

The inventor has discovered simple, highly efficient methods for production of enzymatically active urokinase. The instant methods utilize, in some embodiments, crude bacterially-produced pro-urokinase (e.g., either cell paste or inclusion bodies), and generate correctly folded, highly active pro-urokinase or urokinase using only a small number of steps. The methods of the instant invention generate overall yields of at least 20-40%, and can be used to produce urokinase with activity of at least about 100,000 international units per milligram (IU/mg) of protein. Importantly, the inventor has found that pro-urokinase produced in accordance with the invention is highly stable in solution (as described herein, may be stored at −20° C. for more than 6 months), and so is well suited for use in liquid formulations of urokinase.

Generally, the invention provides methods of producing enzymatically active urokinase or pro-urokinase from pro-urokinase which needs to be properly folded, such as pro-urokinase from inclusion bodies from bacteria cells, by solubilizing, for example, the inclusion body protein in a buffer containing disulfide reducing agents and a high concentration of chaotroph (e.g., 8 M urea or 6 M guanidine HCl) at high pH (i.e., greater than about pH 9), and refolding by reducing the chaotroph concentration and slowly reducing the pH to near-neutral (i.e., pH 7.5-8.5). The refolded pro-urokinase may then be purified. For production of urokinase, refolded pro-urokinase is digested with an appropriate serine protease, such as plasmin or trypsin, either before or after purification of the refolded protein.

The invention provides methods for producing refolded recombinant pro-urokinase by solubilizing denatured or misfolded pro-urokinase protein with a solubilization buffer containing a high concentration of chaotroph, a reducing agent, and having a pH of about 9.0 to about 11.0, to produce a solubilized pro-urokinase solution, rapidly diluting the solubilized pro-urokinase solution with refolding buffer by adding the solubilized pro-urokinase solution into the refolding buffer to produce a diluted solubilized pro-urokinase solution, and reducing the pH of the diluted solubilized pro-urokinase solution to a pH of about 7.5 to about 8.5, wherein said pH reducing is carried out over a period of at least about 20 hours, thereby producing refolded pro-urokinase.

In certain embodiments the pro-urokinase is human pro-urokinase.

In certain embodiments the chaotroph is urea, which may be at about 8 M concentration. In other embodiments, the chaotroph is guanidine hydrochloride, which may be at about 6 M concentration.

In certain embodiments, the solubilizing buffer is about pH 10. In certain embodiments, the solubilizing buffer is about pH 10.5. In certain embodiments, the solubilizing buffer is pH about 10.0 to about 10.5.

In certain embodiments the pH of the diluted solubilized pro-urokinase solution is reduced to about pH 8.0.

In certain embodiments solubilized pro-urokinase solution is diluted into about twenty-fold refolding buffer.

In certain embodiments, the refolding buffer comprises urea at about 0.8 M to about 2.5 M and arginine at about 0.05 M to about 1.5 M. In certain embodiments the refolding buffer comprises urea at about 2.0 M and arginine at about 1 M. In certain embodiments the refolding buffer comprises urea at about 2.0 M and arginine at about 0.2 M. In certain embodiments, the refolding buffer comprises guanidine HCl at about 1 M and arginine at about 0.2 M.

The invention may comprise additional steps at the beginning of the process. Thus, in certain embodiments the method includes the preliminary step of lysing bacterial host cells comprising denatured pro-urokinase protein and collecting said denatured pro-urokinase protein. Certain additional embodiments also include washing the denatured pro-urokinase protein.

The invention may also comprise additional steps at the end of the process. Thus, certain embodiments also include purification of the refolded pro-urokinase, such as by size exclusion chromatography (SEC), ion exchange chromatography (IEC), heparin affinity chromatography, hydroxyapatite chromatography, or a combination of these steps, such as both SEC and IEC, SEC followed by cation exchange chromatography, and SEC followed by heparin affinity chromatography and hydroxyapatite chromatography, which can be used in either order.

In certain embodiments, the invention includes cleaving the refolded pro-urokinase to produce urokinase. The urokinase thus produced may have specific activity of at least about 100,000 international units per milligram (IU/mg) of protein.

In an exemplary embodiment, denatured pro-urokinase polypeptide is solubilized with a buffer comprising about 8 M urea and about 100 mM beta-mercaptoethanol at about pH 10, to produce solubilized pro-urokinase polypeptide, concentration adjusted to about 3.6 mg/mL, rapidly diluted about twenty-fold with refolding buffer comprising about 2 M urea, about 1 M arginine (in some embodiments, the refolding buffer further comprising oxidized and reduced glutathione), and the pH of the diluted solubilized pro-urokinase polypeptide is adjusted to about pH 8 over a period of at least about 24 hours.

In another exemplary embodiment, denatured pro-urokinase polypeptide is solubilized with a buffer comprising about 8 M urea and about 100 mM beta-mercaptoethanol at about pH 10.5, to produce solubilized pro-urokinase polypeptide, concentration adjusted to about 3.6 mg/mL, rapidly diluted about twenty-fold with refolding buffer comprising about 2 M urea, about 0.2 M arginine (in some embodiments, the refolding buffer further comprising oxidized and reduced glutathione), and the pH of the diluted solubilized pro-urokinase polypeptide is adjusted to about pH 8 over a period of at least about 24 hours.

In another exemplary embodiment, denatured pro-urokinase polypeptide is solubilized with a buffer comprising about 8 M urea and about 100 mM beta-mercaptoethanol at about pH 10.5, to produce solubilized pro-urokinase polypeptide, concentration adjusted to about 3.6 mg/mL, rapidly diluted about twenty-fold with refolding buffer comprising about 1 M guanidine HCl, about 0.2 M arginine (in some embodiments, the refolding buffer further comprising oxidized and reduced glutathione), and the pH of the diluted solubilized pro-urokinase polypeptide is adjusted to about pH 8 over a period of at least about 24 hours.

In some embodiments, the solubilized pro-urokinase polypeptide solution is diluted in a buffer containing about 8 M urea, about 10 mM β-mercaptoethanol, about 10 mM dithiothreitol (DTT), about 1 mM reduced glutathion (GSH), and about 0.1 mM oxidized glutathion (GSSG) at about pH 10.0 or 10.5 to adjust the pro-urokinase concentration to about 3.6 mg/ml.

The invention also provides properly folded pro-urokinase and urokinase produced by the instant methods and compositions comprising properly folded pro-urokinase and urokinase.

### BRIEF DESCRIPTION OF THE SEVERAL VIEWS OF THE DRAWING(S)

FIGS. 1a and 1b show the nucleotide [SEQ ID NO:1] sequence of the human pro-urokinase gene used to produce pro-urokinase protein [SEQ ID NO:2] in Examples 1 and 2. Italicized bases in the nucleotide sequence indicate bases which were changed to increase expression efficiency in E. coli. Protein sequence is shown in single letter amino acid code.

FIG. 2 shows a Hanes plot of an urokinase activity assay described in Example 3.

FIG. 3 shows the a synthetic nucleotide sequence [SEQ ID NO:5] encoding human pro-urokinase protein [SEQ ID NO:2] designed to optimize the pro-urokinase gene expression in E. coli.

### DETAILED DESCRIPTION OF THE INVENTION

The instant invention provides simple, efficient methods for the production of recombinant, properly folded pro-urokinase and urokinase.

The practice of the present invention will employ, unless otherwise indicated, conventional techniques of immunology, molecular biology, microbiology, cell biology and recombinant DNA, which are within the skill of the art. See, e.g., Molecular Cloning: a laboratory manual, 2^{nd} edition Sambrook, et al. (1989); Current Protocols In Molecular Biology F. M. Ausubel, et al. eds., (1987); the series Methods In Enzymology, Academic Press, Inc.; PCR 2: A Practical Approach, M. J. MacPherson, B. D. Hames and G. R. Taylor, eds. (1995), and Antibodies, A Laboratory Manual, Harlow and Lane, eds. (1988).

It should be noted that, as used herein, the singular form “a”, “an”, and “the” includes plural references unless indicated otherwise. Additionally, as used herein, in accordance with well-established principles of patent law, the term “comprising” and its cognates are used in their inclusive sense; that is, equivalent to the term “including” and its corresponding cognates.

The methods of the invention are typically practiced utilizing inclusion bodies containing pro-urokinase polypeptide, such as are formed in bacterial (e.g., E. coli) cells which have been engineered to produce pro-urokinase, as the starting material, but any source of denatured or misfolded pro-urokinase protein may be used. The pro-urokinase may be from any species desired, and from any natural or non-natural pro-urokinase sequence, according to the practitioner's preference. See, for example, SEQ ID NO:5. The full coding sequence of a number of pro-urokinase genes are publicly available (e.g., human, rat, mouse, and rabbit pro-urokinase sequences are available from Genbank under the accession numbers NM 002658, NM 013085, NM 008873, and AY122285, respectively), and partial coding sequences which would allow the isolation of the full sequence are available for additional species (e.g., horse, cow, macaque monkey, etc.). Additionally, altered pro-urokinase genes, such as genes with the deletions of sequences encoding the secretion signal sequence, genes with “silent” changes which improve expression in the host organism (“optimized” sequences), or genes encoding mutant pro-urokinase with one or more amino acid sequence changes may also be used.

Examples of non-natural pro-urokinase sequence and mutant pro-urokinase are pro-urokinase mutants that are designed to improve pro-urokinase functionality for therapeutic purposes. For example, pro-urokinase may be mutated to have a lower intrinsic activity than wild type pro-urokinase but have enzymatic activity similar to the wild type pro-urokinase when activated. See, e.g., U.S. Pat. No. 5,472,692; Sun et al., J. Biol. Chem. 272:23818-23823 (1997). These mutants may cause lower non-specific plasminogen activation and bleeding complications than wild type pro-urokinase when administered to a patient. Mutant pro-urokinases that are resistant to natural inhibitors of pro-urokinase and activated urokinase such as plasminogen activator inhibitors (PAI-1 and PAI-2) have been described. See, e.g., Adams et al., J. Biol. Chem. 266:8476-8482 (1991). These mutants may have prolonged half life and activity of pro-urokinase and may have increased therapeutic potency relative to wild type pro-urokinase. Other examples of mutant pro-urokinase are mutants that reduce interaction of pro-urokinase with the urokinase plasminogen activator receptor (uPAR). See, e.g., Arterioscler. Thromb. Vasc. Biol. 18: 693-701 (1998). These mutants may have reduced non-specific activation of pro-urokinase on epithelial cells that line the blood vessel walls while away from blood clot and may be unlikely to cause non-specific systemic bleeding.

Recombinant (e.g., bacterial, such as E. coli) host cells may be engineered to produce pro-urokinase polypeptide using any convenient technology. Any suitable host cell and expression system may be used. Bacterial host cells are exemplified herein. Most commonly, a DNA sequence encoding the desired pro-urokinase is inserted into the appropriate site in a plasmid-based expression vector which provides appropriate transcriptional and translational control sequences, although expression vectors based on bacteriophage genomic DNA are also useful. It is generally preferred that the transcriptional control sequences are inducible by a change in the environment surrounding the host cells (such as addition of a substrate or pseudosubstrate to which the transcriptional control sequences are responsive), although constitutive transcriptional control sequences are also useful. As is standard in the art, it is also preferred that the expression vector include a positive selectable marker (e.g., the β-lactamase gene, which confers resistance to ampicillin) to allow for selection against bacterial host cells which do not contain the expression vector.

The bacterial host cells are typically cultured in a liquid growth medium for production of pro-urokinase polypeptide under conditions appropriate to the host cells and expression vector. Preferably, the host cells are cultured in a bacterial fermenter to maximize production, but any convenient method of culture is acceptable (e.g., shaken flask, especially for cultures of less than a liter in volume). As will be apparent to those of skill in the art, the exact growing conditions, timing and rate of media supplementation, and addition of inducing agent (where appropriate) will vary according to the identity of the host cells and the expression construct.

After the bacterial host cells are cultured to the desired density (and after any necessary induction of expression), the cells are collected. Collection is typically conveniently effected by centrifugation of the growth medium, although any other convenient technique may be used. The collected bacterial host cells may be washed at this stage to remove traces of the growth medium, most typically by resuspension in a simple buffer followed by centrifugation (or other convenient cell collection method). At this point, the collected bacterial host cells (the “cell paste”) may be immediately processed in accordance with the invention, or it may be frozen for processing at a later time.

The cells of the cell paste are lysed to release the pro-urokinase polypeptide-containing inclusion bodies. Preferably, the cells are lysed under conditions in which the cellular debris is sufficiently disrupted that it fails to appear in the pellet under low speed centrifugation. Commonly, the cells are suspended in a buffer at about pH 5 to 9, preferably about 6 to 8, using an ionic strength of the order of about 0.01 M to 2 M preferably about 0. 1-0.2 M (it is apparently undesirable to use essentially zero ionic strength). Any suitable salt, including NaCl can be used to maintain an appropriate ionic strength level. The cells, while suspended in the foregoing buffer, are then lysed by techniques commonly employed such as, for example, mechanical methods such as freeze/thaw cycling, the use of a Manton-Gaulin press, a French press, or a sonic oscillator, or by chemical or enzymatic methods such as treatment with lysozyme. It is generally desirable to perform cell lysis, and optionally bacterial cell collection, under conditions of reduced temperature (i.e., less than about 20° C.).

Inclusion bodies are collected from the lysed cell paste using any convenient technique (e.g., centrifugation), then washed. If desired, the collected inclusion bodies may be washed. Inclusion bodies are typically washed by resuspending the inclusion bodies in a wash buffer, typically the lysis buffer, preferably with a detergent added (e.g., 1% TRITON X-100®, then recollecting the inclusion bodies. The washed inclusion bodies are then dissolved in solubilization buffer. Solubilization buffer comprises a high concentration of a chaotroph, a pH buffer that buffers the solution to a high pH, and one or more reducing reagents. The solubilization buffer may optionally contain additional agents, such as redox reagents, cation chelating agents and scavengers to neutralize protein-damaging free-radicals.

The instant invention utilizes urea as an exemplary chaotroph in the solubilization buffer, although guanidine hydrochloride (guanidine HCl) may also be used. Useful concentrations of urea in the solubilization buffer include about 7.5 M to about 9 M, about 8 M to about 8.5M, or about 8 M. When the chaotroph is guanidine HCl, useful concentrations include about 5 M to about 7 M, or about 5.5 M to about 6.5 M, or about 6 M.

The pH of the solubilization buffer is high, viz., in excess of pH 9.0. Useful pH levels in the solubilization buffer are in the range of about 9.0 to about 11.0, about 9.5 to about 10.5, about 10.0 to about 10.5, about 10, or about 10.5. As will be apparent to those of skill in the art, any pH buffering agent (or combination of agents) which effectively buffer at high pH are useful, although pH buffers which can buffer in the range of about pH 8 to about pH 9 or 10 are particularly useful. Useful pH buffering agents include tris (tris(hydroxymethyl)aminomethane), bicine (N,N-Bis(2-hydroxyethyl)glycine), HEPBS (2-Hydroxy-1,1-bis[bydroxymethyl[ethyl)amino]-1-propanesulfonic acid), TAPS ([(2-Hydroxy-1,1-bis[bydroxymethyl]ethyl)amino]-1-propanesulfonic acid), AMPD (2-Amino-2-methyl-1,3-propanediol).N-(2-Hydroxyethyl)piperazine-N′-(4-butanesulfonic acid)), and the like. The pH buffering agent is added to a concentration that provides effective pH buffering, such as from about 50 to about 150 mM, about 75 mM to about 125 mM, or about 100 mM.

Reducing reagents are included in the solubilization buffer to reduce disulfide bonds and maintain cysteine residues in their reduced form. Useful reducing reagents include β-mercaptoethanol, dithiothreitol, and the like. Additionally, the solubilization buffer may contain disulfide reshuffling or “redox” reagents (e.g., a combination of oxidized and reduced glutathione). When the redox reagents are oxidized and reduced glutathione (GSSG and GSH, respectively), the inventor has found that useful concentrations include about 0.1 mM to about 11 mM and useful ratios include about 10:1, about 5:1, and about 1:1 (GSSG:GSH).

The solubilization buffer may contain additional components. For example, the solubilization buffer may contain a cation chelator such as a divalent cation chelator like ethylenediaminetetraacetic acid (EDTA) or ethylene glycol-bis(2-aminoethylether)-N,N,N′,N′-tetraacetic acid(EGTA). EDTA or EGTA is added to the solubilization buffer at a concentration of about 0.5 to about 5 mM, and commonly at about 1 mM. Additionally, a free-radical scavenger may be added to reduce or eliminate free-radical-mediated protein damage, particularly if urea is used as the chaotroph and it is expected that a urea-containing protein solution will be stored for any significant period of time. Suitable free-radical scavengers include glycine (e.g., at about 0.5 to about 2 mM, or about 1 mM) and other amino acids and amines.

The inclusion body/solubilization buffer mixture is incubated to allow full solubilization. The incubation period is generally from about six hours to about 24 hours, and more commonly about eight to about 14 hours or about 12 hours. The inclusion body/solubilization buffer mixture incubation may be carried out at reduced temperature, commonly at about 4° to about 10° C.

After the incubation is complete, the inclusion body/solubilization buffer mixture is clarified to remove insoluble debris. Clarification of the mixture may be accomplished by any convenient means, such as filtration (e.g., by use of depth filtration media) or by centrifugation. Clarification should be carried out at reduced temperature, such as at about 4° to about 100° C.

The clarified mixture is then diluted to achieve the appropriate protein concentration for refolding. Protein concentration may be determined using any convenient technique, such as Bradford assay, light absorption at 280 nm (A₂₈₀), and the like. The inventor has found that a solution having an A₂₈₀ of about 2.0 to about 10.0 is appropriate for use in the instant methods, such as about 5.0 (approximately 3.6 mg/mL). The A₂₈₀ of the solution may be adjusted to any of about 5.0 to about 10.0, about 2.0 to about 8.0, about 2.0 to about 5.0, or less than any of about 8.0, about 5.0, about 4.0, about 3.0, or any of about 2.0, 5.0, 6.0, 8.0, 10.0. If desired, this mixture may be held, refrigerated (e.g. at 4° C.), for later processing, although the mixture is not normally held for more than about four weeks. In some embodiments, the solubilized pro-urokinase polypeptide solution is diluted in a buffer comprising about 8 M urea, about 10 mM β-mercaptoethanol, about 10 mM dithiothreitol (DTT), about 1 mM reduced glutathion (GSH), and about 0.1 mM oxidized glutathion (GSSG) at pH of about 9.0 to about 11.0. In some embodiments, the pH of the buffer is about 10.0 to about 10.5. In some embodiments, the pH is about 10.0. In other embodiments, the pH is about 10.5. In some embodiments, the buffer comprises 8 M urea, 100 mM tris, 1 mM glycine, 10 mM β-mercaptoethanol, 10 mM dithiothreitol (DTT), 1 mM reduced glutathion (GSH), and 0.1 mM oxidized glutathion (GSSG) at pH of about 10.0.

The concentration-adjusted inclusion body solution is first rapidly diluted about 20 fold with refolding buffer. The dilution is performed by adding inclusion body solution into the refolding buffer. The inclusion body solution may be diluted about 10 to about 100 fold, about 10 to about 50 fold, about 10 to about 25 fold, about 15 to about 25 fold with refolding buffer. The inclusion body solution is diluted to reduce urea and protein concentration. The final protein concentration after dilution may be about 0.01 mg/ml to about 1 mg/ml, about 0.1 mg/ml to about 0.5 mg/ml. The refolding buffer generally contains a low concentration of chaotroph, a pH buffer, and a divalent cation chelator. The refolding buffer may also contain a disulfide reshuffling reagent. The refolding buffer may include additional agents, such as free-radical scavengers and detergents. “Rapid” dilution, within the context of the invention means over a period of less than about 25 minutes, and the dilution process is generally carried out during periods of about two minutes to about 25 minutes, or about five to about 20 minutes. The diluted solubilized pro-urokinase solution is typically held for one to two hours following the completion of the rapid dilution process.

Chaotrophs useful in the refolding buffer include urea, guanidine, and arginine, although the inventor has found that urea alone is ineffective. However, the inventor has found that a combination of the urea and arginine is efficacious in the refolding buffer. When a combination of urea and arginine is utilized, the urea may be from about 0.8 to about 2.5 M, about 0.9 to about 2 M, about 1 M to about 2.5 M, or about 2.0 or 1.0 M, and the arginine may be from about 0.05 M to about 1.5 M, about 0.2 M to about 1.0 M, about 0.5 M to about 1.5 M, about 0.75 M to about 1.25 M, about 0.2 M, or about 1 M. The inventor has also found that a combination of guanidine HCl and arginine is efficacious in the refolding buffer. When a combination of guanidine and arginine is utilized, the guanidine HCl may be from about 1 mM to about 1.5 M, or about 0.05 M to about 1.5 M, or about 1 M, and the arginine may be from about 0.05 M to about 1.5 M, about 0.2 M to about 1.0 M, about 0.5 M to about 1.5 M, about 0.75 M to about 1.25 M, about 0.2 M, or about 1 M.

The pH buffering agent in the refolding buffer may be any buffering agent or combination of buffering agents that are effective pH buffers at pH levels of about 8 to about 9 or 10. Useful pH buffering agents include tris (tris(hydroxymethyl)aminomethane), bicine (N,N-Bis(2-hydroxyethyl)glycine), HEPBS (2-Hydroxy-1,1-bis[bydroxymethyl]ethyl)amino]-1-propanesulfonic acid), TAPS ([(2-Hydroxy-1,1-bis[bydroxymethyl]ethyl)amino]-1-propanesulfonic acid), and AMPD (2-Amino-2-methyl-1,3-propanediol).N-(2-Hydroxyethyl)piperazine-N′-(4-butanesulfonic acid)). The pH buffering agent is added to a concentration that provides effective pH buffering, such as from about 50 to about 150 mM, about 75 mM to about 125 mM, or about 100 mM.

The redox reagents included in the refolding buffer must be effective in ‘shuffling’ cysteine sulfhydryl groups between their oxidized and reduced states. The redox environment of the refolding reaction may be adjusted by manipulating the concentration of the redox reagents. When the redox reagents are oxidized and reduced glutathione (GSSG and GSH, respectively), the inventor has found that useful concentrations include about 0.1 mM to about 11 mM and useful ratios include about 10:1, about 5:1, and about 1:1 (GSSG:GSH).

The divalent cation chelator may be any molecule that effectively chelates Ca⁺⁺ and other divalent cations. Exemplary cation chelators for use in the refolding buffer include ethylenediaminetetraacetic acid (EDTA) or ethylene glycol-bis(2-aminoethylether)-N,N,N′,N′-tetraacetic acid(EGTA). When EDTA or EDTA is the divalent cation chelator, it is added to the refolding buffer at a concentration of about 0.5 to about 5 mM, and commonly at about 1 mM.

Additional components useful in the refolding buffer include free-radical scavengers and detergents. A free-radical scavenger may be added to reduce or eliminate free-radical-mediated protein damage, particularly if urea is used as the chaotroph and it is expected that a urea-containing protein solution will be stored for any significant period of time. Suitable free-radical scavengers include glycine (e.g., at about 0.5 to about 2 mM, or about 1 mM). Low concentrations of detergents may also be added to the refolding buffer, such as about 0.01% to about 0.1% TWEEN® 20.

The pH of the refolding solution is then slowly reduced from elevated pH to near neutral pH using an appropriate acid. The time period for pH reduction can range from about 20-24 hours to about 10 days, about 20 to about 50 hours, about 20 to about 40 hours, about 20 to about 30 hours, about 24 to about 40 hours. The time period for pH reduction can be at least any of about 20 hours, about 24 hours, about 30 hours, about 40 hours, about 50 hours. Appropriate acids for pH adjustment will depend on the pH buffer used in the refolding buffer. For example, when the pH buffering agent is tris, the pH should be adjusted with hydrochloric acid (HCl).

Following completion of pH adjustment, the refolding reaction is incubated for a period of about one to two hours to about 18 to 24 hours. The refolding reaction may be carried out at room temperature (e.g., about 18-20° C.) or at slightly reduced temperatures (e.g., about 14-16° C.), depending on the preferences of the practitioner and the available facilities.

Following the refolding reaction, properly refolded pro-urokinase may be concentrated, further purified, and/or the protein may be proteolytically processed (e.g., with plasmin) to produce mature urokinase. Concentration of the refolded protein may be accomplished using any convenient technique, such as ultrafiltration, diafilitration, chromatography (e.g., ion-exchange, hydrophobic interaction, or affinity chromatography) and the like. The concentration step may also include a buffer exchange process, if so desired. Where practical, it is preferred that concentration be carried out at reduced temperature (e.g., about 4-10° C.).

While any convenient purification protocol may be used, the inventor has utilized a simple procedure which utilizes two chromatographic steps, an initial size-exclusion chromatography step and a final ion exchange chromatography step. In some embodiments, a heparin affinity chromatography is used for purification. The heparin affinity chromatography may involve binding of the refolded protein to immobilized heparin. In other embodiments, a hydroxyapatite chromatography is used for purification. The hydroxyapatite chromatography may be used following a heparin affinity chromatography. As with concentration (and optional buffer exchange), the purification step(s) may be carried out at reduced temperature, such as 4° C.

Size exclusion chromatography (SEC) may be performed using any convenient chromatography medium which separates properly folded pro-urokinase from unfolded pro-urokinase and multimeric pro-urokinase. The inventor has found that media having the ability to size fractionate proteins of about 10⁴ to about 6×10⁵ daltons (globular proteins) are useful for this step. Exemplary SEC media include Sephacryl® 300 and Superdex™ 200. This step may also be used to perform buffer exchange, if so desired. The exact conditions for SEC will depend on the exact chromatography media selected, whether buffer exchange is to be accomplished, the requirements of any later purification steps, and other factors known to those of skill in the art.

The properly folded pro-urokinase may be further purified utilizing cation exchange chromatography. An exemplary cation exchange chromatography step utilizes an exchange resin derivatized with sulfopropyl groups (a strong cation exchanger). The column is loaded in low salt conditions, washed under low salt conditions, and eluted in elevated salt conditions. When guanidine HCl is used as the chaotroph, the material from the SEC column should be diluted to reduce the guanidine HCl concentration to less than about 0.2 M. As with SEC, the exact conditions for IEC will depend on the exact chromatography media selected, whether buffer exchange is to be accomplished, the requirements of any later purification steps, and other factors known to those of skill in the art. Generally, loading conditions will have low ionic strength and a lower pH (e.g., pH about 6 to 7), and high concentrations of chaotroph should be avoided.

The properly refolded pro-urokinase may be further purified with heparin affinity chromatography and/or hydroxyapatite chromatography. Heparin affinity chromatography may be performed with any immobilized heparin. Exemplary media include Heparin Hyper™ M, HiTrap™ Heparin HP and the like. Exemplary media for hydroxyapatite chromatography is CHT-II hydroxyapatite (BioRad).

In some embodiments, the refolded pro-urokinase (in refolding buffer) is diluted 5 fold with 25 mM HEPES pH 7.0, and loaded directly on a column with immobilized heparin such as heparin-sepharose (Pharmacia/Amersham). Before loading of the refolded pro-urokinase, the column is first equilibrated in a low salt buffer with no chelators or primary or secondary amines, for example, 50 mM Hepes, 25 mM NaCl, pH 7.0. After the pro-urokinase is bound to the column, the column is washed with 10 column volumes of the buffer used for equilibration. Next, the column is washed with the same buffer with the NaCl concentration raised to 125 mM in order to elute impurities. Finally, pro-urokinase is eluted form the column with 50 mM Hepes, 500 mM NaCl, pH 7.0. Buffers used for heparin affinity chromatography may optionally include 0.1 mM bezamidine. Fractions from the column that contain pro-urokinase are pooled and applied to a hydroxyapatite column. Before loading the hydroxyapatite column is first equilibrated in 50 mM HEPES, pH 7.0, 150 mM NaCl. After loading the pro-urokinase, the column is washed with the equilibration buffer until no more protein elutes. The column is then washed in 50 mM HEPES pH 7.0, 150 mM NaCl, 100 mM sodium phosphate pH 7.0; washing is continued until no more protein elutes. Finally, pro-urokinase is eluted from the column with a buffer containing 50 mM HEPES pH 7.0, 150 mM NaCl, 500 mM sodium phosphate pH 7.0.

The purified refolded pro-urokinase can be stored at 4° C. or −20° C. in solution. In some embodiments, the pro-urokinase is stored in a buffer containing about 20 mM tris, about 0.2 M NaCl, and about 10-20% glycerol at about pH 7.4. The inventor has observed that pro-urokinase generated in accordance with the invention can be stored at −20° C. in 20 mM tris, 0.2 M NaCl, and 20% glycerol, pH 7.4 for more than six months.

Urokinase may be produced from the refolded pro-urokinase produced by the instant invention utilizing any method known in the art, such as plasmin digestion. Plasmin processing of pro-urokinase is well known in the art, and may be performed using any convenient method. Example 3, herein, describes one useful method utilizing plasmin (incubation with plasmin (0.1 μg/mL) for 60 minutes at 37° C., followed by addition of an excess of protease inhibitor such as 12,500 IU/mL of aprotinin). Alternately, the pro-urokinase may be processed to urokinase using plasmin bound to a solid phase.

Activity of urokinase produced from the properly folded recombinant pro-urokinase produced in accordance with the invention may be measured using any acceptable assay method. An exemplary method of measuring urokinase activity is the method used in Example 3, herein, which utilizes activation of the pro-urokinase to urokinase by plasmin digestion, followed by measurement of urokinase activity using the chromogenic substrate S-2444 (Wang et al., 2000, Thromb. Res. 100:461-67; Orsini et al., 1991, Eur. J. Bioch. 195:691-97).

As is well understood in the art, all concentrations and pH values need not be exact and reference to a given value reflects standard usage in the art, does not mean that the value cannot vary.

The invention also provides compositions comprising pro-urokinase or urokinase produced by the methods described herein. The invention also provides pharmaceutical compositions comprising an effective amount of pro-urokinase or urokinase produced by the methods of the invention, and a pharmaceutically acceptable carrier. Pharmaceutically acceptable carriers are known in the art.

The following examples provide a detailed description of the production of properly folded recombinant pro-urokinase in accordance with the methods of the invention and the characterization thereof. These examples are not intended to limit the invention in any way.

### EXAMPLES

### Example 1

Refolding and Purification of Recombinant Pro-Urokinase

### Example 1A.

A DNA fragment encoding human pro-urokinase was produced by PCR amplification of a kidney cDNA library using primers UK-1 (5′-CATATGTCCAACGAACTGCACCAGGTTCCATCGAACTGTGACTGTC-3′ [SEQ ID NO:3]) and UK-2 (5′-CTCGAGTTAGAGGGCCAGGCCATTCTCTTC-3′ [SEQ ID NO:4]). Primer UK-1 was designed to introduce 6 silent mutations into the pro-urokinase gene that increase the efficiency of expression in E. coli.

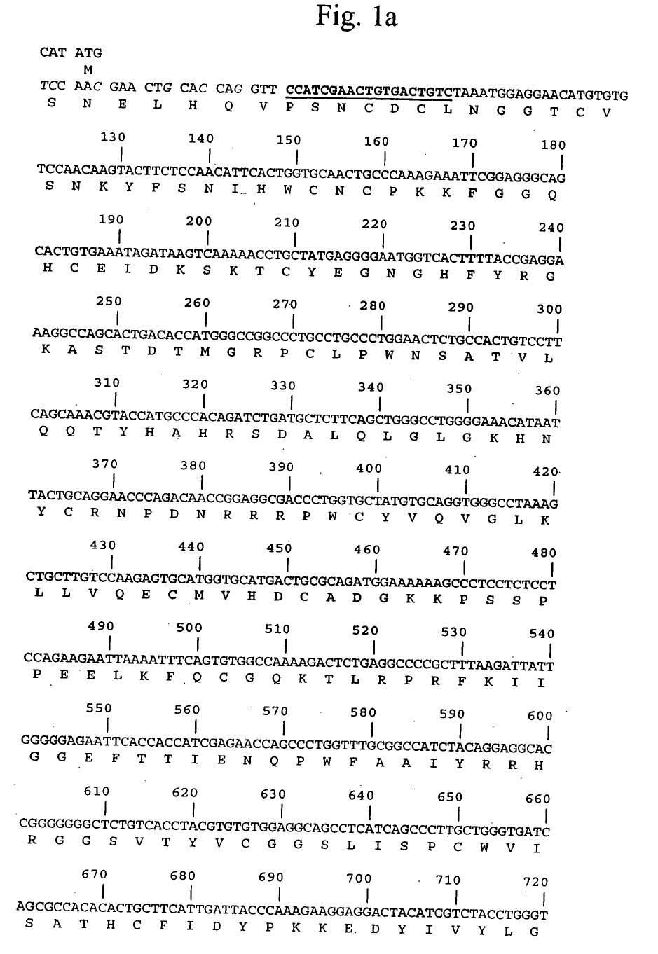

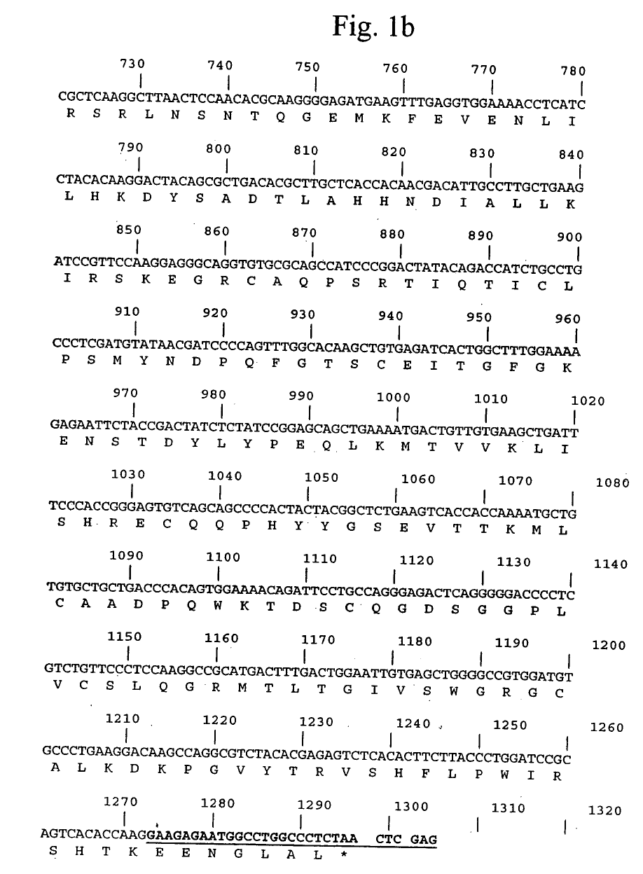

The full-length PCR product was cloned into pCR2.1TOPO (Invitrogen) and sequenced from both ends using M13F and M13R primers. The nucleotide and encoded protein sequences are shown in FIG. 1. The insert was excised by NdeI-XhoI restriction digested, gel purified, then cloned into NdeI-XhoI digested pET43 (Novagen).

The pro-urokinase expression vector was transfected into BL21 (DE3) strain of E. coli and plated on ZB plates with ampicillin. A single colony was selected and used to inoculate 100 mL of ZB media (10 g/l NZ amine A (Sigma) and 5 g/l NaCl) with ampicillin and grown overnight (approximately 16 hours) at 37° C. The 20 mL of the 100 mL starter culture was then used to inoculate 1 L of ZB media with ampicillin, and the culture was incubated at 37° C. with shaking until the optical density at 600 nm (OD₆₀₀) reached 0.4-0.6. Isopropyl-beta-D-thiogalactopyranoside (IPTG) was then added to 0.5 mM to induce pro-urokinase expression, and the culture was incubated a further three hours with shaking.

Cells were collected by centrifugation, then resuspended in 20 mL of TN (150 mM NaCl, 50 mM tris, pH 8.0) with 1% TRITON X-100®. Ten milligrams of lysosyme was added, and the cell suspension was frozen at −20° C. overnight. The lysate was then thawed and 20 μL of 1 M magnesium sulfate and 100 μg of DNase was added. The cells were incubated, with stirring, until the released bacterial DNA was completely dissolved.

The lysate was then diluted with 250 mL of TN with 1% TRITON X-100® and the mixture was stirred for 2-4 hours. Inclusion bodies were collected by centrifugation, and washed three times (by resuspension and centrifugation).

The washed inclusion bodies were dissolved in 10 mL of urea solution (8M urea, 100 mM tris, 1 mM glycine, pH 10), and β-mercaptoethanol (BME) was added to 100 mM. The solution was clarified by ultracentrifugation (30 minutes×66,000 g), then refolded. In another experiment, the urea solution was pH 10.5.

Refolding was performed by first rapidly diluting the clarified solution with (by adding the clarified solution into) 20 volumes of 20 mM tris, 0.2 M arginine, 1 M guanidine HCl, 1 m M EDTA, initially adjusting the pH to 10, then gradually reducing the pH to 8.0. The pH reduction was carried out by reducing the pH by 0.2 pH units every four hours using 6 M HCl.

The refolded product was concentrated by ultrafiltration (Millipore Pellicon, 10,000 Da cut-off membrane), then subjected to SEC using a SEPHACRYL® S-300 column equilibrated with 10 mM tris, 0.4 M urea, 1 M guanidine HCl, 0.2 M arginine, and 1 mM EDTA, pH 8.0.

Additional purification was obtained by IEC, using a SP Sepharoase FF column equilibrated with phosphate buffer and eluted with a zero to 1 M NaCl gradient.

The final product was stored refrigerated (4° C.) and frozen (−20° C.) in 20 mM tris, 0.4 M urea, 1 mM EDTA, and 20% glycerol, pH 8.0.

### Example 1B

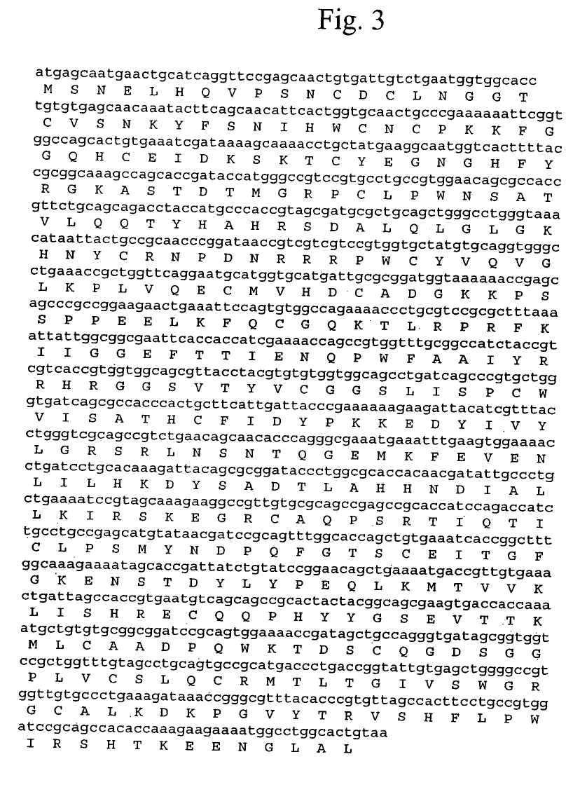

A synthetic nucleotide sequence [SEQ ID NO:5] (shown in FIG. 3) encoding human pro-urokinase was also used for expressing the human pro-urokinase protein. This synthetic nucleotide sequence was designed to optimize the gene expression in E. coli by optimizing codon usage in E. coli expression and taking consideration of RNA secondary structures.

### Example 2

Purification of Recombinant Pro-Urokinase by Heparin Affinity Chromatography and Hydroxyapatite Chromatography

The refolded pro-urokinase produced as described in Example 1 was concentrated by ultrafiltration (Millipore Pellicon, 10,000 Da cut-off membrane), then subjected to SEC using a SEPHACRYL® S-300 column equilibrated with 10 mM tris, 0.4 M urea, 1 M guanidine HCl, 0.2 M arginine, and 1 mM EDTA, pH 8.0. By this chromatography, the refolded pro-urokinase was separated from misfolded high molecular weight aggregate.

Fractions from the S-300 column containing the pro-urokinase were pooled, diluted 5 fold with 25 mM HEPES pH 7.0, and applied to an affinity column of HiTrap™ Heparin HP sepharose (Pharmacia/Amersham). The heparin affinity column was first equilibrated in 25 mM HEPES pH 7.0, 25 mM NaCl. After loading the sample, the column was washed with the equilibration buffer until no further protein was detected in the eluate. The column was then washed with 25 mM HEPES pH 7.0, 125 mM NaCl until no further protein was detected in the eluate. The pro-urokinase was then eluted with a buffer containing 25 mM HEPES pH 7.0, 500 mM NaCl. Fractions containing pro-urokinase were then pooled and applied directly to a BioRad CHT II hydroxyapatite column. The hydroxyapatite column had been equilibrated in 50 mM HEPES pH 7.0, 100 mM NaCl, 10 mM sodium phosphate pH 7.0. After loading the pro-urokinase, the column was washed in the equilibration buffer until no more protein could be detected in the elulate. The column was then washed with 50 mM HEPES pH 7.0, 100 mM NaCl, 100 mM sodium phosphate pH 7.0 until no protein was detected in the eluate. The pro-urokinase was then eluted from the column with a buffer containing 50 mM HEPES pH 7.0, 100 mM NaCl, 500 mM sodium phosphate pH 7.0. The pooled fractions containing the pro-urokinase were then dialyzed into 50 mM HEPES pH 7.0, 150 mM NaCl, 0.1 mM bezamidine, 10% (v/v) glycerol and stored at 4° C.

### Example 3

Characterization of Recombinant Urokinase

Purified human pro-urokinase, produced as described in Example 1, was diluted to 1 μM in 50 mM tris, 50 mM NaCl, 0.01% TWEEN® 20, pH 8.9, and converted to urokinase by incubation with plasmin (0.1 μg/mL) for 60 minutes at 37° C. The reaction was stopped by addition of excess (12,500 IU/mL) aprotinin.

Enzymatic activity was measured by diluting the urokinase 10-fold and incubating with various concentrations of the chromagenic substrate S-2444 (pyro-Glu-Gly-Arg-pNA, DiaPharma) and measuring the production of free pNA by monitoring absorbance at 405 nm (A₄₀₅). Results were plotted using a Hanes plot. A sample of a urokinase international standard (obtained from NIBSC/WHO) was run in parallel to allow for calculation of activity in international units (IU).

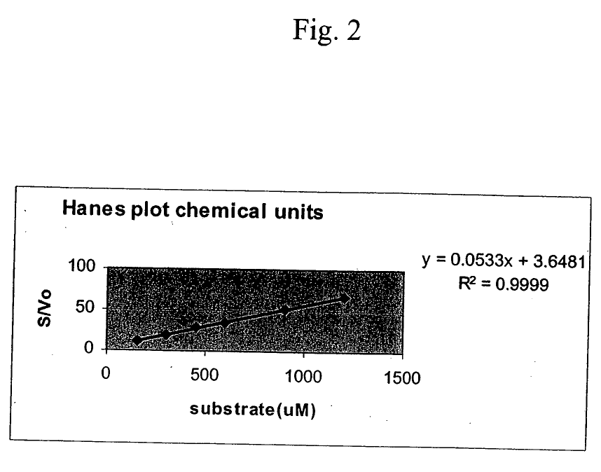

The Hanes plot is shown in FIG. 2. The purified urokinase had activity of 100,000 IU/mg (±9.5%), Kcat/Km (min μM)⁻¹ of 2.9, Kcat (min)⁻¹ of 196, and Km(μM) of 68.

### Example 4

Refolding, Purification and Characterization of a Mutant Human Pro-Urokinase

A mutant human pro-urokinase which has Lys³⁰⁰ substituted by His (K300H) was produced and refolded. Substituting Lys³⁰⁰ by His for pro-urokinase is described in U.S. Pat. No. 5,472,692 and Sun et al., J. Biol. Chem. 272:23818-23823 (1997).

The DNA fragment encoding human pro-urokinase shown in FIG. 1 was used for site-directed mutagenesis to replace Lys³⁰⁰ with His. The mutagenesis was accomplished using standard PCR techniques employing the Stratagene QuikChange® Site-Directed Mutagenesis Kit and primers 5′ atcactggctttggacacgagaattctaccgac3′ [SEQ ID NO:6] and 5′ gtcggtagaattctcgtgtccaaagccagtgat3′ [SEQ ID NO:7]. The DNA fragment was cloned into expression vector pCR2.1 TOPO (Invitrogen) and sequenced from both ends as described in Example 1. The insert was then excised by NdeI-XhoI restriction digested, gel purified, then cloned into NdeI-XhoI digested pET43 (Novagen).

The expression vector encoding the mutant pro-urokinase was transfected into E. coli and inclusion bodies containing mutant pro-urokinase K300H were generated as described in Example 1. Mutant pro-urokinase K300H from the inclusion bodies was then refolded. Briefly, the washed inclusion bodies were dissolved in 10 mL of urea solution (8M urea, 100 mM tris, 1 mM glycine, pH 10), and then diluted in 8M urea, 100 mM tris, 1 mM glycine, 10 mM beta-mercaptoethanol, 10 mM DTT, 1 mM reduced glutathion (GSH), 0.1 mM oxidized glutathion (GSSG), pH 10.0 until the A₂₈₀ of the solution reached 2.0. Refolding was then performed by first rapidly diluting the clarified solution with (by adding the clarified solution into) 20 volumes of 20 mM tris, 0.2 M arginine, 1 M guanidine HCl, 1 m M EDTA, initially adjusting the pH to 10, then gradually reducing the pH to 8.0. The pH reduction was carried out by reducing the pH by 0.2 pH units every four hours using 6 M HCl.

The refolded product was purified using methods described in Example 1. An additional purification step utilizing p-amino benzamidine affinity chromatography was included subsequent to the purification steps described in Example 1 to remove any trace of activated urokinase from the pro-urokinase. To accomplish this, NaCl was added to pro-urokinase stored in 20 mM tris, 0.4 M urea, 20% glycerol, pH 8.0 to raise the NaCl concentration to 0.5M . The pro-urokinase was then loaded onto the p-amino benzamidine column (purchased from Sigma) pre-equilibrated in identical buffer. Only pro-urokinase that did not bind to the p-aminobenzamidine column was utilized for kinetic characterization. Purified mutant human pro-urokinase and wild type pro-urokinase(proUK) were diluted to 1 μM in 50 mM tris, 50 mM NaCl, 0.01% TWEEN® 20, pH 8.9 and were tested for their kinetic parameters using substrate S-2444 (pyro-Glu-Gly-Arg-pNA, DiaPharma) and measuring the production of free pNA by monitoring absorbance at 405 nm (A₄₀₅) at 21° C. as described in Example 3. The kinetic parameters were determined using a Hoftsee plot. Table 1 shows the kinetic parameters of wild type proUK and mutant K300H that have not been activated by plasmin.

TABLE 1
Comparison of kinetic parameters for wild type proUK and
mutant K300H

| Type of proUK | Km (μM) | Kcat (min⁻¹) | Kcat/Km (μM min)⁻¹ |
|---|---|---|---|
| Wild type proUK | 135 | 0.158 | 1.17 × 10⁻³ |
| K300H | 130 | 0.040 | 3.07 × 10⁻⁴ |

The kinetic parameters of the wild type proUK and mutant K300H were also determined after they were activated by plasmin and converted to urokinase as described in Example 3. Table 2 shows the kinetic parameters of plasmin-activated wild type proUK and mutant K300H.

TABLE 2
Comparison of kinetic parameters for plasmin-activated wild
type proUK and mutant K300H

| Type of proUK | Km (μM) | Kcat (min⁻¹) | Kcat/Km (μM min)⁻¹ |
|---|---|---|---|
| Activated wild | 27.9 | 250 | 8.96 |
| type proUK | | | |
| Activated K300H | 22.5 | 218 | 9.69 |

As shown in Table 1, the catalytic efficiency (Kcat/Km) for non-activated (zymogenic) mutant K300H was 26% less efficient as compared to non-activated wild type proUK. When mutant K300H is activated, it was more catalytically efficient (108%) than the activated wild type proUK.

Although the foregoing invention has been described in some detail by way of illustration and example for purposes of clarity and understanding, it will be apparent to those skilled in the art that certain changes and modifications may be practiced. Therefore, descriptions and examples should not be construed as limiting the scope of the invention, which is delineated by the appended claims.

I claim:

1. A method of producing refolded recombinant pro-urokinase, comprising:
(a) solubilizing pro-urokinase protein with a solubilization buffer, said solubilization buffer comprising a high concentration of chaotroph, a reducing agent, and having a pH of about 9.0 to about 11.0, thereby producing a solubilized pro-urokinase solution;
(b) rapidly diluting said solubilized pro-urokinase solution with refolding buffer by adding said solubilized pro-urokinase solution into the refolding buffer, thereby producing a diluted solubilized pro-urokinase solution;
(c) reducing the pH of the diluted solubilized pro-urokinase solution to a pH of about 7.5 to about 8.5, wherein said pH reducing is carried out over a period of at least about 20 hours, thereby producing refolded pro-urokinase.

2. The method of claim 1, wherein said pro-urokinase is human pro-urokinase.

3. The method of claim 1, wherein said chaotroph is urea.

4. The method of claim 3, wherein said urea is at about 8 M concentration.

5. The method of claim 1, wherein said chaotroph is guanidine hydrochloride.

6. The method of claim 5, wherein said guanidine hydrochloride is at about 6 M concentration.

7. The method of claim 1, wherein said solubilization buffer is pH about 10.0 to about 10.5.

8. The method of claim 1, wherein the pH of the diluted solubilized pro-urokinase solution is reduced to about pH 8.0.

9. The method of claim 1, said method further comprising adjusting the A₂₈₀ of the solubilized pro-urokinase solution to about 2.0 to about 5.0 before step (b).

10. The method of claim 9, wherein the A₂₈₀ of the solubilized pro-urokinase solution is adjusted by diluting the solubilized pro-urokinase solution in a buffer comprising about 8 M urea, about 10 mM β-mercaptoethanol, about 10 mM dithiothreitol (DTT), about 1 mM reduced glutathion (GSH), and about 0.1 mM oxidized glutathion (GSSSG) at pH about 10 to about 10.5.

11. The method of claim 1, wherein the solubilized pro-urokinase solution is diluted into about twenty-fold refolding buffer.

12. The method of claim 1, wherein the refolding buffer comprises urea at about 0.8 M to about 2.5 M and arginine at about 0.05 M to about 1.5 M.

13. The method of claim 12, wherein said refolding buffer comprises urea at about 2.0 M and arginine at about 0.2 M.

14. The method of claim 1, wherein the refolding buffer comprises guanidine HCl at about 1 M and arginine at about 0.2 M.

15. The method of claim 1, wherein said solubilization buffer comprises about 8 M urea and about 100 mM beta-mercaptoethanol and has a pH of about 10.5; said solubilized pro-urokinase solution is adjusted to A₂₈₀ of about 5.0 then rapidly diluted into about 20 fold refolding buffer, and said refolding buffer comprises about 2 M urea, about 0.2 M arginine.

16. The method of claim 1, wherein said solubilization buffer comprises about 8 M urea and about 100 mM beta-mercaptoethanol and has a pH of about 10.5; said solubilized pro-urokinase solution is adjusted to A₂₈₀ of about 5.0 then rapidly diluted into about 20 fold refolding buffer, and said refolding buffer comprises about 1 M guanidine HCl, about 0.2 M arginine.

17. The method of claim 1, further comprising lysing bacterial host cells comprising denatured pro-urokinase protein and collecting said denatured pro-urokinase protein.

18. The method of claim 17, further comprising washing said denatured pro-urokinase protein.

19. The method of claim 1, further comprising purifying said refolded pro-urokinase.

20. The method of claim 19, wherein said refolded pro-urokinase is purified by size exclusion chromatography (SEC).

21. The method of claim 19, wherein said refolded pro-urokinase is purified by ion exchange chromatography (IEC).

22. The method of claim 19, wherein said refolded pro-urokinase is purified by heparin affinity chromatography.

23. The method of claim 19, wherein said refolded pro-urokinase is purified by hydroxyapatite chromatography.

24. A composition comprising refolded pro-urokinase, produced by the method of claim 1.
